(19) United States
(12) Patent Application Publication
(10) Pub. No.: US 2022/0395439 A1
(43) Pub. Date: Dec. 15, 2022

(54) ANTIBACTERIAL DEODORANT COMPOSITION AND PRODUCTION METHOD THEREFOR

(71) Applicant: MEDIPOOL CO., LTD., Gyeongsan-si, Gyeongsangbuk-do (KR)

(72) Inventor: Ho Cheong KIM, Daegu (KR)

(21) Appl. No.: 17/777,158

(22) PCT Filed: Nov. 22, 2019
(86) PCT No.: PCT/KR2019/016104

§ 371 Date: May 16, 2022

(30) Foreign Application Priority Data
Nov. 22, 2019 (KR) 10-2019-0151107

(51) Int. Cl. A61K 8/19; A61K 8/02; A01N 59/20; A61Q 15/00

(52) U.S. Cl. CPC A61K 8/19; A61K 8/0241; A01N 59/20; A61Q 15/00

(57) ABSTRACT

The antibacterial deodorant composition according to the present invention includes an aqueous colloidal solution containing 1,500-2,500 ppm of nonionic copper nanoparticles having an average diameter (D50) of 2-10 nm.

### TECHNICAL FIELD

The present invention relates to an antibacterial deodorant composition and a production method therefor.

### BACKGROUND ART

A bad odor refers to an unpleasant or disgusting odor caused by irritation of the human sense of smell from pungent gaseous substances such as hydrogen sulfide, mercaptans, amines, ammonia, and the like.

Methods to deal with the odor include a ventilation dilution method that diffuses the odor into the air by means of a hood or duct, a perfume-use camouflage method that disguises the odor by dispersing ingredients with a strong aroma, an absorption method that absorbs and removes odorous substances by passing the substances through a washer containing charcoal or cleaning liquid, a condensation method that condenses the odorous substances using a cooler, a combustion oxidation method that directly burns the odorous substances with a flame of 600° C. to 800° C., and a biological deodorization method using microorganisms, and the like.

Among the above-described methods, the ventilation dilution method or the perfume-use camouflage method has the advantage of having the lowest cost, but cannot be a fundamental countermeasure since these methods do not remove causative substances of the odor, and the absorption method, the condensation method, and the combustion oxidation method have the disadvantage of high cost.

On the other hand, the biological deodorization method has the advantages of providing an excellent deodorizing effect and having low facility and maintenance costs even if it is installed in an area where odor-causing substances are generated on a large scale, but still has the disadvantage in that it is possible only when the causative substance of the odor is larger than a certain scale.

When removing odors from apartments or offices, it is difficult to apply the biological deodorization method that is able to be applied only when there are odor-causing substances over a certain scale or the combustion oxidation method that generates unpleasant by-products, and the like, and it is difficult to use the absorption method with very low odor removal efficiency or the condensation method that requires a cooler over a certain size. Thus, inevitably, the ventilation dilution method or the perfume-use camouflage method is mainly used.

However, as described above, since the ventilation dilution method or the perfume-use camouflage method is not a fundamental solution, improvement measures that are able to be applied to small places such as apartments and offices by supplementing other deodorization methods are constantly being studied.

### DISCLOSURE

### Technical Problem

An object of the present invention is to provide an antibacterial deodorant composition capable of resolving a fundamental odor by having excellent deodorizing effect as well as excellent antibacterial effect, and a production method therefor.

All of the above-described and other objects of the present invention can be achieved by the present invention described below.

### Technical Solution

In one general aspect, there is provided an antibacterial deodorant composition.

The antibacterial deodorant composition may include an aqueous colloidal solution containing 1,500 to 2,500 ppm of nonionic copper nanoparticles having an average particle diameter (D50) of 2 nm to 10 nm.

The aqueous colloidal solution may further include 20 to 200 ppm of silver nanoparticles.

The copper nanoparticles and the silver nanoparticles may have a concentration ratio of 10:1 to 50:1.

The antibacterial deodorant composition may further include one or more of 0.1 to 5 parts by weight of pyrethrin and 1 to 10 parts by weight of an auxiliary solvent based on 100 parts by weight of the aqueous colloidal solution.

The antibacterial deodorant composition may further include one or more of 0.001 to 0.1 parts by weight of a titanium dioxide catalyst and 0.001 to 0.1 parts by weight of zinc oxide based on 100 parts by weight of the aqueous colloidal solution.

The titanium dioxide catalyst may contain titanium dioxide (TiO₂), copper (Cu), and magnesium (Mg), wherein the copper (Cu) and magnesium (Mg) in the titanium dioxide catalyst may be contained in a content of 5 to 50% by weight.

The copper (Cu) and magnesium (Mg) may have a weight ratio of 10:90 to 20:80.

In another general aspect, there is provided a production method for an antibacterial deodorant composition.

The production method for the antibacterial deodorant composition may include adding 1 to 6 mol of sodium hydroxide (NaOH) per 1 mol of copper chloride (CuCl₂) to an aqueous solution of copper chloride (CuCl₂) to generate copper oxide and copper hydroxide in the solution; and adding 1 to 12 mol of hydrazine (N₂H₄) per 1 mol of copper chloride (CuCl₂) to the generated copper oxide and copper hydroxide to perform reduction to nonionic copper nanoparticles, thereby producing an aqueous colloidal solution containing copper nanoparticles.

Further, the copper nanoparticles may have an average particle diameter (D50) of 2 nm to 10 nm, and may be contained at a concentration of 1,500 ppm to 2,500 ppm in the aqueous colloidal solution.

### Advantageous Effects

The present invention provides an antibacterial deodorant composition capable of resolving a fundamental odor by having excellent deodorizing effect as well as excellent antibacterial effect, and a production method therefor.

### DESCRIPTION OF DRAWINGS

FIG. 1 is a scanning electron microscope (SEM) image of nonionic copper nanoparticles contained in an antibacterial deodorant composition of Example 1.

### BEST MODE

Hereinafter, the present invention will be described in more detail.

In describing the present invention, when it is determined that a detailed description of well-known technology associated with the present invention may unnecessarily make unclear the gist of the present invention, the detailed description thereof will be omitted.

When terms ‘including’, ‘having’, ‘constituting’, and the like, described in the present specification are used, other parts may be added unless ‘merely’ is used. Although a component is used in a singular form, it may include a plural form unless explicitly described to the contrary.

In addition, in analyzing the components, it is construed as including an error range even if there is no separate explicit description.

Further, in the present specification, the phrase ‘X to Y’ expressing the range means ‘more than X and less than Y’.

Antibacterial Deodorant Composition

An antibacterial deodorant composition according to an embodiment of the present invention includes an aqueous colloidal solution containing 1,500 to 2,500 ppm of nonionic copper nanoparticles having an average particle diameter (D50) of 2 nm to 10 nm.

The antibacterial deodorant composition of the present invention has an excellent deodorizing effect by including the aqueous colloidal solution containing copper nanoparticles.

The aqueous colloidal solution containing 1,500 to 2,500 ppm of nonionic copper nanoparticles having an average particle diameter (D50) of 2 nm to 10 nm may be produced by a production method for an antibacterial deodorant composition to be described later.

The copper particles)(Cu⁰) that are not ionized and have a size of 2 nm to 10 nm (hereinafter, referred to as nonionic copper nanoparticles) not only have excellent deodorizing and antibacterial effect, but also do not cause any side effects to the human body. If the average particle diameter (D50) of the copper nanoparticles is beyond the range of the present invention, there is a high risk of remaining as impurities after using the antibacterial deodorant composition.

In addition, when the concentration of the nonionic copper nanoparticles contained in the aqueous colloidal solution is less than 1,500 ppm, the deodorizing and antibacterial effects are insignificant, and when the concentration of the nonionic copper nanoparticles contained in the aqueous colloidal solution is more than 2,500 ppm, there is a possibility that the copper nanoparticles may remain as impurities.

The aqueous colloidal solution may further contain silver nanoparticles. The silver nanoparticles have the advantage of interfering with the metabolism of bacteria by an antibacterial action to suffocate bacteria, having a strong bactericidal effect, and having the effect of strengthening human immunity, and thus it is possible to maintain the deodorizing effect of the antibacterial deodorant composition for a long period of time.

The silver nanoparticles may have an average particle diameter (D50) of 50 nm to 150 nm, specifically 70 nm to 100 nm. In the above particle diameter range, the antibacterial effect is sufficiently exhibited without reducing other effects.

The silver nanoparticles may be contained at a concentration of 20 to 200 ppm in the aqueous colloidal solution. In the above content range, it is possible to maximize the antibacterial action without compromising the deodorizing effect.

The aqueous colloidal solution may contain the copper nanoparticles and the silver nanoparticles in a concentration ratio of 10:1 to 50:1, specifically 15:1 to 40:1. In the above concentration ratio range, the balance of the antibacterial effect and the deodorizing effect is excellent.

In another embodiment, the antibacterial deodorant composition may further contain one or more of pyrethrin and an auxiliary solvent.

The pyrethrin may further improve the antibacterial effect as well as the deodorizing effect.

The pyrethrin (CAS No. 121-21-1, (1S)-2-methyl-4-oxo-3-[(2Z)-2,4-pentadien-1-yl]-2-cyclopenten-1-yl (1R, 3R)-2,2-dimethyl-3-(2-methyl-1-propen-1-yl)cyclopropanecarboxylate) is a natural insecticidal component extracted from petals of the Asteraceae family and has a high antibacterial effect. Since the pyrethrin has no effect on humans and animals and is rapidly decomposed, it is possible to resolve problems caused by the residual. The pyrethrin may be used as a commercially available product under the trade name of Pyrethrum, but is not limited thereto.

The pyrethrin may be contained in a content of 0.1 to 5.0 parts by weight, specifically 0.3 to 2 parts by weight, based on 100 parts by weight of the aqueous colloidal solution containing the nonionic copper nanoparticles. In the above content range, it is possible to exhibit a sufficient antibacterial effect, and to prevent the particles from remaining as impurities.

The auxiliary solvent may include at least one of methanol, ethanol, propanol, butanol, ethyl acetate, propylene glycol, and butylene glycol. Specifically, as the auxiliary solvent, ethanol may be used in consideration of compatibility with the aqueous colloidal solution and solubility of other components. In particular, the pyrethrin has low solubility in water but high solubility in ethanol, and thus when ethanol is applied, there is an effect of improving the compatibility of pyrethrin.

The auxiliary solvent may be contained in a content of 1 to 10 parts by weight, specifically 3 to 8 parts by weight, based on 100 parts by weight of the aqueous colloidal solution. In the above content range, it is possible to maximize the compatibility of the components of the composition, and to provide a disinfecting effect.

The antibacterial deodorant composition may contain pyrethrin and the auxiliary solvent in a weight ratio of 1:3 to 1:7, specifically 1:4 to 1:6. In the above content range, the antibacterial effect and compatibility are able to be maximized.

In still another embodiment, the antibacterial deodorant composition may further contain one or more of a titanium dioxide catalyst and zinc oxide.

The titanium dioxide (TiO₂) catalyst is a catalyst that generates electrons (e) in the conduction band (CB) and holes (h) in the valence band (VB) by light of a specific wavelength. The titanium dioxide catalyst may further enhance the antibacterial and deodorizing effect.

In an embodiment, the titanium dioxide catalyst may contain titanium dioxide (TiO₂), copper (Cu), and magnesium (Mg), wherein the copper (Cu) and magnesium (Mg) in the titanium dioxide catalyst may be contained in a content of 5 to 50% by weight.

The titanium dioxide may have an average particle diameter (D50) of 1 nm to 100 nm, specifically 5 nm to 80 nm.

In addition, the titanium dioxide may include two or more kinds of titanium dioxide having different average particle diameters (D50). In this case, catalyst efficiency according to the compactness of the catalyst is improved.

For example, the titanium dioxide (TiO₂) may contain first and second titanium dioxide (TiO₂) having different average particle diameters (D50), wherein the first titanium dioxide (TiO₂) may have an average particle diameter (D50) of 1 nm to 70 nm, specifically 10 nm to 50 nm, and the second titanium dioxide (TiO₂) may have an average particle diameter (D50) of 20 nm to 100 nm, specifically 20 nm to 80 nm.

An average particle diameter (D50) ratio of the first titanium dioxide (TiO₂) and the second titanium dioxide (TiO₂) may be 1:0.4 to 1:0.6. In the above average particle diameter ratio range, the titanium dioxide catalyst may be more dense and may maximize the catalyst efficiency.

The titanium dioxide (TiO₂) may be contained in a content of 50 to 95% by weight, specifically 60 to 90% by weight of the titanium dioxide catalyst. In the above content range, sufficient catalytic efficiency may be exhibited, and at the same time, other effects may also be exerted.

The copper (Cu) and magnesium (Mg) may be contained in the catalyst component to increase the light absorption rate in the visible light region, thereby not only improving the catalytic efficiency, but also exhibiting catalytic activity more easily, and thus it is possible to maximize catalyst efficiency.

In particular, in the titanium dioxide catalyst of the present invention, the weight ratio of copper (Cu) and magnesium (Mg) is applied in the range of 10:90 to 20:80, specifically 12:88 to 16:84, so as to be close to the eutectic point. Thus, the titanium dioxide catalyst is produced at a remarkably low temperature. This may minimize the transition to the rutile crystal structure, thereby maximizing the catalyst efficiency. In this case, the copper (Cu) and magnesium (Mg) may be eutectic or more. Here, the copper (Cu) and magnesium (Mg) components may serve to improve the bonding force between the titanium dioxide catalyst components, and to mold the titanium dioxide catalyst to a specific particle diameter range. Specifically, the particle diameter of the titanium dioxide catalyst may be determined depending on the content of the copper (Cu) and magnesium (Mg) components.

The copper (Cu) and magnesium (Mg) may be contained in a content of 5 to 50% by weight, specifically 10 to 45% by weight, and more specifically 15 to 40% by weight of the titanium dioxide catalyst. In the above content range, the titanium dioxide catalyst may not only improve the catalytic efficiency in the visible region and the catalytic efficiency at a lower current, but also minimize the side effect of discoloration by the titanium dioxide catalyst.

The titanium dioxide catalyst may be produced by a process including forming a mixture of titanium dioxide (TiO₂) powder, copper (Cu), and magnesium (Mg), and performing heat treatment on the mixture. The heat treatment may be performed at 400° C. to 900° C., specifically 450° C. to 750° C., and more specifically 460° C. to 550° C. in a H₂/Ar atmosphere. Copper (Cu) and magnesium (Mg) contained in the titanium dioxide catalyst of the present invention may be applied in a weight ratio of 10:90 to 20:80, and may be eutectic at least in part in the heat treatment temperature range, and thus it is possible to sufficiently add a bonding force between the components constituting the catalyst. In particular, the content of the copper (Cu) and magnesium (Mg) components may affect the particle diameter of the titanium dioxide catalyst. Specifically, copper (Cu) and magnesium (Mg) may be contained in a content of 5 to 50% by weight of the titanium dioxide catalyst to control the average particle diameter (D50) of the titanium dioxide catalyst to 1 mm to 10 mm.

The titanium dioxide catalyst may be contained in a content of 0.001 to 0.1 parts by weight, specifically 0.005 to 0.05 parts by weight, based on 100 parts by weight of the aqueous colloidal solution. In the above content range, the antibacterial effect and deodorizing effect of the antibacterial deodorant composition may be maximized.

The zinc oxide (ZnO) is a white to yellowish fine amorphous powder, has no smell and taste, and has a property of slowly absorbing carbon dioxide from the air.

The zinc oxide imparts antibacterial and bactericidal power through a mechanism that kills and eliminates viruses or bacteria by inhibiting the metabolism thereof, and in particular, has a strong deodorizing effect among metals while performing a metal catalyst function.

The zinc oxide may be contained in a content of 0.001 to 0.1 parts by weight, specifically 0.005 to 0.05 parts by weight, based on 100 parts by weight of the aqueous colloidal solution. In the above content range, it is possible to have sufficient antibacterial and deodorizing effect and to prevent an increase in production cost.

Production Method for Antibacterial Deodorant Composition

A production method for the antibacterial deodorant composition according to an embodiment of the present invention may include adding 1 to 6 mol of sodium hydroxide (NaOH) per 1 mol of copper chloride (CuCl₂) to an aqueous solution of copper chloride (CuCl₂) to generate copper oxide and copper hydroxide in the solution; and adding 1 to 12 mol of hydrazine (N₂H₄) per 1 mol of copper chloride (CuCl₂) to the generated copper oxide and copper hydroxide to perform reduction to nonionic copper nanoparticles, thereby producing an aqueous colloidal solution containing copper nanoparticles.

In the present invention, copper chloride (CuCl₂) is used as a precursor of copper nanoparticles. Copper chloride (CuCl₂), unlike copper sulfate (CuSO₄), has an anionic functional group with relatively high electronegativity, thereby achieving an anionic effect different from that of sulfate ion in the solution, and thus it is possible to further suppress a phenomenon that the produced particles aggregate with each other. Therefore, it is possible to manufacture finer particles, and to exhibit an excellent effect of controlling a surface shape.

In the step of adding sodium hydroxide (NaOH) to the aqueous solution of copper chloride (CuCl₂) to generate copper oxide and copper hydroxide in the solution is a step of adding sodium hydroxide (NaOH) to an aqueous solution of copper chloride (CuCl₂), thereby producing copper oxide (CuO) as an intermediate and copper hydroxide (Cu(OH)₂) as a complex compound, which may be represented by Chemical Reaction Scheme 1 below:

3CuCl₂+6NaOH→CuO+6Na⁺+6Cl⁻+Cu₂O+Cu(OH)₂+2H₂O [Chemical Reaction Scheme 1]

In Chemical Reaction Scheme 1, sodium hydroxide (NaOH) is added to separate chlorine from copper atoms of copper chloride (CuCl₂) to produce copper oxide and copper hydroxide, wherein the sodium hydroxide (NaOH) may be added in the range of 1 to 6 mol per 1 mol. When the content of sodium hydroxide to be added is more than 6 mol, the condition in the solution changes to a strong basicity, and thus the reduction reaction of hydrazine to be added later does not occur smoothly, and it is not economical because a lot of unreacted substances are generated, and further impurities increase due to a large number of residual ions in the solution. On the other hand, when the content of sodium hydroxide (NaOH) to be added is less than 1 mol, the form of copper oxide (CuₓO), which is an intermediate, is not completely formed, and thus it is difficult to smoothly perform the reaction.

A temperature of the aqueous copper chloride (CuCl₂) solution to which the sodium hydroxide (NaOH) is added is preferably adjusted to be in the range of 25° C. to 60° C. When the temperature of the aqueous copper chloride solution is less than 25° C., it is difficult to form an intermediate. When the temperature of the aqueous copper chloride solution is more than 60° C., the intermediate may be generated too quickly, which may cause aggregation of the intermediate, and the reduction reaction proceeds at an excessively high temperature, which may reduce thermal stability of the intermediate.

In the step of adding hydrazine (N₂H₄) to the produced copper oxide and copper hydroxide to perform reduction to copper nanoparticles, hydrazine (N₂H₄) may be added to reduce copper oxide (CuO) produced as the intermediate and the complex compound copper hydroxide (Cu(OH)₂), thereby producing copper nanoparticles (Cu°) precipitated in a nonionic state, which may be represented by Chemical Reaction Scheme 2 below:

CuO+6Na⁺+6Cl⁻+Cu₂O+Cu(OH)₂+2N₂H₄→3Cu+6Na+6Cl⁻+4 H₂O+2N₂ [Chemical Reaction Scheme 2]

The content of hydrazine (N₂H₄) added in the above Chemical Reaction Formula 2 is in the range of 1 to 12 mol per 1 mol of copper chloride, wherein when hydrazine is added in a content of less than 1 mol, it is difficult to perform the reduction reaction completely, and when hydrazine is added in a content of more than 12 mol, the reduction reaction occurs at a fast rate due to the use of excess hydrazine, but the aggregation phenomenon of the obtained copper nanoparticles may occur severely.

A temperature of the aqueous solution to which the hydrazine (N₂H₄) is added is preferably maintained in the range of 35° C. to 60° C. When the temperature of the aqueous solution to be added is less than 35° C., not only the reaction rate of the reduction reaction is low, but also a conversion rate of the reduction reaction is low, and thus complete reduction may not be achieved. On the other hand, when the temperature of the aqueous solution to be added is more than 60° C., the reaction rate of the reduction reaction may be slightly increased, but the reaction proceeds at a high temperature, and the aggregation phenomenon of the produced copper nanoparticles may occur severely.

In addition, the copper nanoparticles may have an average particle diameter (D50) of 2 nm to 10 nm, and may be contained at a concentration of 1,500 ppm to 2,500 ppm in the aqueous colloidal solution.

In another embodiment, in the production method for the antibacterial deodorant composition, 20 to 200 ppm of silver nanoparticles may be further contained in the aqueous colloidal solution. The silver nanoparticles may have an average particle diameter (D50) of 50 nm to 150 nm, specifically 70 nm to 100 nm. In the above particle diameter range, the antibacterial effect is sufficiently exhibited without reducing other effects.

In still another embodiment, the production method for the antibacterial deodorant composition may further include adding, based on 100 parts by weight of the aqueous colloidal solution, one or more of 0.1 to 5 parts by weight of pyrethrin, 1 to 10 parts by weight of an auxiliary solvent, 0.001 to 0.1 parts by weight of a titanium dioxide catalyst, and 0.001 to 0.1 parts by weight of zinc oxide to the aqueous colloidal solution, followed by mixing.

The pyrethrin, auxiliary solvent, titanium dioxide catalyst, and zinc oxide are substantially the same as those described in the antibacterial deodorant composition according to one aspect of the present invention.

Hereinafter, the constitution and function of the present invention will be described in more detail through preferred embodiments of the present invention. However, these are presented as preferred examples of the present invention and cannot be construed as limiting the present invention in any sense.

Descriptions that are not provided in the present specification will be omitted since they can be technically inferred sufficiently by those skilled in the art.

### EXAMPLE

### Example 1

100 ml of 2M aqueous copper chloride (CuCl₂) solution was prepared and stirred vigorously while heating, wherein a temperature was maintained at 35° C. When the temperature of the aqueous copper chloride solution was kept constant in the above temperature range, 6M sodium hydroxide (NaOH) was added at once. After sodium hydroxide (NaOH) was added, 15M hydrazine (N₂H₄) was added at once while maintaining the temperature of the solution at 45° C. to reduce copper particles to obtain an aqueous colloidal solution, thereby producing an antibacterial deodorant composition. The copper nanoparticles in the aqueous colloidal solution had a concentration of about 2,000 ppm.

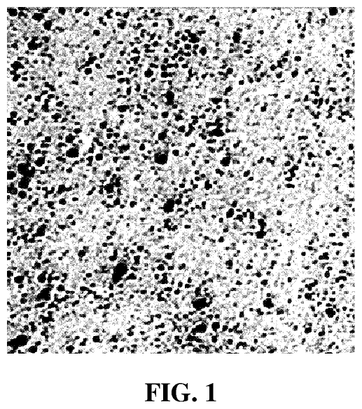

An average particle diameter (D50) of the copper nanoparticles contained in the finally produced antibacterial deodorant composition was measured to be 3 nm. FIG. 1 is a scanning electron microscope (SEM) image of the produced copper nanoparticles.

### Example 2

An antibacterial deodorant composition was produced in the same manner as in Example 1, except that 100 ppm of silver nanoparticles having an average particle diameter (D50) of 100 nm were added to the aqueous colloidal solution obtained in Example 1.

### Example 3

An antibacterial deodorant composition was produced in the same manner as in Example 2, except that 1 part by weight of pyrethrin was further added to the antibacterial deodorant composition of Example 2.

### Example 4

An antibacterial deodorant composition was produced in the same manner as in Example 2, except that 1 part by weight of pyrethrin together with 5 parts by weight of ethanol used as an auxiliary solvent was further added to the antibacterial deodorant composition of Example 2.

### Example 5

An antibacterial deodorant composition was produced in the same manner as in Example 2, except that 0.01 parts by weight of a titanium dioxide catalyst was further added to the antibacterial deodorant composition of Example 2.

The titanium dioxide catalyst was produced by mixing 80% by weight of titanium dioxide (TiO₂, Aldrich), 2.8% by weight of copper (Cu), and 17.2% by weight of magnesium (Mg), putting the mixture into a tube furnace, heating the mixture at 530° C. for 5 hours under H₂/Ar atmosphere, followed by stirring in 1.0 M HCl solution for 24 hours, washing the resulting product with water to remove acid, and drying the product. Here, the titanium dioxide catalyst had an average particle diameter (D50) of 5.2 mm.

### Example 6

An antibacterial deodorant composition was produced in the same manner as in Example 5, except that 1 part by weight of pyrethrin together with 5 parts by weight of ethanol used as an auxiliary solvent was further added to the antibacterial deodorant composition of Example 5.

### Comparative Example 1

A commercially available antibacterial deodorant from Company E was purchased and used as Comparative Example 1. The antibacterial deodorant was described as containing water, a deodorant, a stabilizer, a surfactant (betaine-based less than 5%), and a fragrance.

### Experimental Method

1) Deodorizing Effect Test

The samples of antibacterial deodorant compositions of Examples and Comparative Example each having a content of 20 mL were placed in a 5 L-sized reactor and sealed. A test gas (trimethylamine) was injected at an initial concentration of 50 μmol/mol. Then, the concentration of the test gas (trimethylamine) was measured at the initial time (0 min), 30 min, 60 min, 90 min, and 120 min, and these measured concentrations were referred to as sample concentrations. The concentrations of the test gas (trimethylamine) were measured by a gas detection plate (KS12218). During the test, the temperature was maintained at 23° C. and the humidity was maintained at 50% R.H.

Separately from this test, the same test as above was performed except that there were no samples, wherein concentrations without the samples were referred to as blank concentrations. A removal rate of the test gas (trimethylamine) for each time period was calculated by Equation 1 below, and was shown in Table 1 below.

Test gas removal rate (deodorization rate)(%)=((blank concentration)−(sample concentration)/(blank concentration))×100 [Equation 1]

2) Antibacterial Effect Test

Antibacterial and bactericidal effects of the antibacterial deodorant compositions in Examples and Comparative Examples were measured. The measurement was carried out by sufficiently applying each composition onto the toilet bowl using the reagent rod and measuring the bacterial count with a bacteria meter (Clean-Q, Model TBD 1000, Teltron Inc.), and results thereof are shown in Table 2 below. The initial bacterial count in the toilet bowl was 1873.

TABLE 1

| | | Blank | Sample | |
|---|---|---|---|---|
| | | Concentration | Concentration | Deodorization |
| | Time | (μmol/mol) | (μmol/mol) | rate (%) |
| Example 1 | 0 min | 50 | 50 | 0 |
| | 30 min | 49 | 8 | 83.7 |
| | 60 min | 49 | 6 | 87.8 |
| | 90 min | 49 | 5 | 89.8 |
| | 120 min | 48 | 4 | 91.7 |
| Example 2 | 0 min | 50 | 50 | 0 |
| | 30 min | 49 | 6 | 87.8 |
| | 60 min | 49 | 4 | 91.8 |
| | 90 min | 49 | 3 | 93.9 |
| | 120 min | 48 | 2 | 95.8 |
| Example 3 | 0 min | 50 | 50 | 0 |
| | 30 min | 49 | 6 | 87.8 |
| | 60 min | 49 | 3 | 93.9 |
| | 90 min | 49 | 3 | 93.9 |
| | 120 min | 48 | 2 | 95.8 |
| Example 4 | 0 min | 50 | 50 | 0 |
| | 30 min | 49 | 4 | 91.8 |
| | 60 min | 49 | 3 | 93.9 |
| | 90 min | 49 | 2 | 95.9 |
| | 120 min | 48 | 1 | 97.9 |
| Example 5 | 0 min | 50 | 50 | 0 |
| | 30 min | 49 | 4 | 91.8 |
| | 60 min | 49 | 2 | 95.9 |
| | 90 min | 49 | 2 | 95.9 |
| | 120 min | 48 | 1 | 97.9 |
| Example 6 | 0 min | 50 | 50 | 0 |
| | 30 min | 49 | 2 | 95.9 |
| | 60 min | 49 | 1 | 98.0 |
| | 90 min | 49 | 1 | 98.0 |
| | 120 min | 48 | 0 | 100 |
| Comparative | 0 min | 50 | 50 | 0 |
| Example 1 | 30 min | 49 | 15 | 69.4 |
| | 60 min | 49 | 11 | 77.6 |
| | 90 min | 49 | 10 | 79.6 |
| | 120 min | 48 | 9 | 81.3 |

TABLE 2

| | | | | | | | Comparative |
|---|---|---|---|---|---|---|---|
| | Example 1 | Example 2 | Example 3 | Example 4 | Example 5 | Example 6 | Example 1 |
| Bacterial | 180 | 163 | 158 | 141 | 136 | 113 | 1332 |
| Count | | | | | | | |

As shown in Tables 1 and 2 above, it could be appreciated that the antibacterial deodorant compositions containing the nonionic copper nanoparticles of the present invention were excellent in not only the antibacterial effect but also the deodorizing effect, whereas Comparative Example 1 without containing the nonionic copper nanoparticles of the present invention showed the antibacterial effect and the deodorizing effect that are inferior to those of the present invention.

Although the embodiments of the present invention have been described above, the present invention is not limited to the above embodiments, but may be manufactured in various different forms. Those of ordinary skill in the art to which the present invention pertains will understand that the present invention may be implemented in other specific forms without changing the technical spirit or essential features of the present invention. Therefore, it should be understood in all respects that the embodiments described above are illustrative and not restrictive.

1. An antibacterial deodorant composition comprising: an aqueous colloidal solution containing 1,500 to 2,500 ppm of nonionic copper nanoparticles having an average particle diameter (D50) of 2 nm to 10 nm.

2. The antibacterial deodorant composition of claim 1, wherein the aqueous colloidal solution further comprises 20 to 200 ppm of silver nanoparticles.

3. The antibacterial deodorant composition of claim 2, wherein the copper nanoparticles and the silver nanoparticles have a concentration ratio of 10:1 to 50:1.

4. The antibacterial deodorant composition of claim 1, further comprising: one or more of 0.1 to 5 parts by weight of pyrethrin and 1 to 10 parts by weight of an auxiliary solvent based on 100 parts by weight of the aqueous colloidal solution.

5. The antibacterial deodorant composition of claim 1, further comprising: one or more of 0.001 to 0.1 parts by weight of a titanium dioxide catalyst and 0.001 to 0.1 parts by weight of zinc oxide based on 100 parts by weight of the aqueous colloidal solution.

6. A production method for the antibacterial deodorant composition according to claim 1, comprising:
adding 1 to 6 mol of sodium hydroxide (NaOH) per 1 mol of copper chloride (CuCl₂) to an aqueous solution of copper chloride (CuCl₂) to generate copper oxide and copper hydroxide in the solution; and
adding 1 to 12 mol of hydrazine (N₂H₄) per 1 mol of copper chloride (CuCl₂) to the generated copper oxide and copper hydroxide to perform reduction to nonionic copper nanoparticles, thereby producing an aqueous colloidal solution containing copper nanoparticles.

7. The production method of claim 6, wherein the copper nanoparticles have an average particle diameter (D50) of 2 nm to 10 nm, and are contained at a concentration of 1,500 ppm to 2,500 ppm in the aqueous colloidal solution.
